SEQUENCE LISTING (77 sequences)

SEQ ID NO: 1 - DNA, 14 bp, Artificial Sequence
  Reference decoy molecule
ctagatttcccgcg

SEQ ID NO: 2 - DNA, 14 bp, Artificial Sequence
  Reference decoy molecule
taaagggcgcctag

SEQ ID NO: 3 - DNA, 18 bp, Artificial Sequence
  Novel decoy molecule
ctagatttcccgcggatc

SEQ ID NO: 4 - DNA, 18 bp, Artificial Sequence
  Novel decoy molecule
gatctaaagggcgcctag

SEQ ID NO: 5 - DNA, 14 bp, Artificial Sequence
  Scrambled decoy
tccagcttcgtagc

SEQ ID NO: 6 - DNA, 13 bp, Artificial Sequence
  Scrambled decoy
gaaggatcgatcg

SEQ ID NO: 7 - DNA, 8 bp, Artificial Sequence
  E2F decoy
tttsgcgs

SEQ ID NO: 8 - DNA, 8 bp, Artificial Sequence
  E2F decoy
tttggcgc

SEQ ID NO: 9 - DNA, 8 bp, Artificial Sequence
  E2F decoy
tttcgcgc

SEQ ID NO: 10 - DNA, 8 bp, Artificial Sequence
  E2F decoy
tttcccgc

SEQ ID NO: 11 - DNA, 8 bp, Artificial Sequence
  E2F decoy
tttgccgc

SEQ ID NO: 12 - DNA, 8 bp, Artificial Sequence
  E2F decoy
cttcccgc

SEQ ID NO: 13 - DNA, 8 bp, Artificial Sequence
  E2F decoy
gttcccgc

SEQ ID NO: 14 - DNA, 8 bp, Artificial Sequence
  E2F decoy
cttcgcgc

SEQ ID NO: 15 - DNA, 8 bp, Artificial Sequence
  E2F decoy
ttagcgcc

SEQ ID NO: 16 - DNA, 8 bp, Artificial Sequence
  E2F decoy
tgagcgcc

SEQ ID NO: 17 - DNA, 8 bp, Artificial Sequence
  E2F decoy
gtagcgcc

SEQ ID NO: 18 - DNA, 8 bp, Artificial Sequence
  E2F decoy
ggagcgcc

SEQ ID NO: 19 - DNA, 8 bp, Artificial Sequence
  E2F decoy
ctagcgcc

SEQ ID NO: 20 - DNA, 8 bp, Artificial Sequence
  E2F decoy
cgagcgcc

SEQ ID NO: 21 - DNA, 8 bp, Artificial Sequence
  E2F decoy
gttcgcgc

SEQ ID NO: 22 - DNA, 8 bp, Artificial Sequence
  E2F decoy
tttgcgcc

SEQ ID NO: 23 - DNA, 8 bp, Artificial Sequence
  E2F decoy
tgtgcgcc

SEQ ID NO: 24 - DNA, 8 bp, Artificial Sequence
  E2F decoy
gttgcgcc

SEQ ID NO: 25 - DNA, 8 bp, Artificial Sequence
  E2F decoy
ggtgcgcc

SEQ ID NO: 26 - DNA, 8 bp, Artificial Sequence
  E2F decoy
cttgcgcc

SEQ ID NO: 27 - DNA, 8 bp, Artificial Sequence
  E2F decoy
cgtgcgcc

SEQ ID NO: 28 - DNA, 7 bp, Artificial Sequence
  E2F decoy
tttccgg

SEQ ID NO: 29 - DNA, 8 bp, Artificial Sequence
  E2F decoy
tttcgcgg

SEQ ID NO: 30 - DNA, 8 bp, Artificial Sequence
  E2F decoy
gttggcgc

SEQ ID NO: 31 - DNA, 8 bp, Artificial Sequence
  E2F decoy
cttggcgc

SEQ ID NO: 32 - DNA, 8 bp, Artificial Sequence
  E2F decoy
cttgccgc

SEQ ID NO: 33 - DNA, 8 bp, Artificial Sequence
  E2F decoy
gttgccgc

SEQ ID NO: 34 - DNA, 8 bp, Artificial Sequence
  E2F decoy
tttggcgg

SEQ ID NO: 35 - DNA, 8 bp, Artificial Sequence
  E2F decoy
ttaccgcc

SEQ ID NO: 36 - DNA, 8 bp, Artificial Sequence
  E2F decoy
tgaccgcc

SEQ ID NO: 37 - DNA, 8 bp, Artificial Sequence
  E2F decoy
gtaccgcc

SEQ ID NO: 38 - DNA, 8 bp, Artificial Sequence
  E2F decoy
ggaccgcc

SEQ ID NO: 39 - DNA, 8 bp, Artificial Sequence
  E2F decoy
ctaccgcc

SEQ ID NO: 40 - DNA, 8 bp, Artificial Sequence
  E2F decoy
cgaccgcc

SEQ ID NO: 41 - DNA, 8 bp, Artificial Sequence
  E2F decoy
tttccgcc

SEQ ID NO: 42 - DNA, 8 bp, Artificial Sequence
  E2F decoy
tgtccgcc

SEQ ID NO: 43 - DNA, 8 bp, Artificial Sequence
  E2F decoy
gttccgcc

SEQ ID NO: 44 - DNA, 8 bp, Artificial Sequence
  E2F decoy
ggtccgcc

SEQ ID NO: 45 - DNA, 8 bp, Artificial Sequence
  E2F decoy
cttccgcc

SEQ ID NO: 46 - DNA, 8 bp, Artificial Sequence
  E2F decoy
cgtccgcc

SEQ ID NO: 47 - DNA, 8 bp, Artificial Sequence
  E2F decoy
cttcccgg

SEQ ID NO: 48 - DNA, 8 bp, Artificial Sequence
  E2F decoy
tttgccgg

SEQ ID NO: 49 - DNA, 8 bp, Artificial Sequence
  E2F decoy
gttcccgg

SEQ ID NO: 50 - DNA, 8 bp, Artificial Sequence
  E2F decoy
cttcgcgg

SEQ ID NO: 51 - DNA, 8 bp, Artificial Sequence
  E2F decoy
tttgcgcg

SEQ ID NO: 52 - DNA, 8 bp, Artificial Sequence
  E2F decoy
ttagcgcg

SEQ ID NO: 53 - DNA, 8 bp, Artificial Sequence
  E2F decoy
tgtgcgcg

SEQ ID NO: 54 - DNA, 8 bp, Artificial Sequence
  E2F decoy
tgagcgcg

SEQ ID NO: 55 - DNA, 8 bp, Artificial Sequence
  E2F decoy
gttgcgcg

SEQ ID NO: 56 - DNA, 8 bp, Artificial Sequence
  E2F decoy
gtagcgcg

SEQ ID NO: 57 - DNA, 8 bp, Artificial Sequence
  E2F decoy
ggtgcgcg

SEQ ID NO: 58 - DNA, 9 bp, Artificial Sequence
  E2F decoy
tttsgcgcg

SEQ ID NO: 59 - DNA, 8 bp, Artificial Sequence
  E2F decoy
cttggcgg

SEQ ID NO: 60 - DNA, 8 bp, Artificial Sequence
  E2F decoy
gttggcgg

SEQ ID NO: 61 - DNA, 8 bp, Artificial Sequence
  E2F decoy
gttcgcgg

SEQ ID NO: 62 - DNA, 8 bp, Artificial Sequence
  E2F decoy
tttcccgg

SEQ ID NO: 63 - DNA, 8 bp, Artificial Sequence
  E2F decoy
cttgccgg

SEQ ID NO: 64 - DNA, 8 bp, Artificial Sequence
  E2F decoy
gttgccgg

SEQ ID NO: 65 - DNA, 8 bp, Artificial Sequence
  E2F decoy
tgtcgcgc

SEQ ID NO: 66 - DNA, 8 bp, Artificial Sequence
  E2F decoy
cttcccgg

SEQ ID NO: 67 - DNA, 8 bp, Artificial Sequence
  E2F decoy
cgtcgcgc

SEQ ID NO: 68 - DNA, 8 bp, Artificial Sequence
  E2F decoy
ggtcgcgc

SEQ ID NO: 69 - DNA, 8 bp, Artificial Sequence
  E2F decoy
tttcgggc

SEQ ID NO: 70 - DNA, 8 bp, Artificial Sequence
  E2F decoy
tgtggcgc

SEQ ID NO: 71 - DNA, 8 bp, Artificial Sequence
  E2F decoy
tgtcgcgg

SEQ ID NO: 72 - DNA, 10 bp, Artificial Sequence
  E2F decoy core sequence
atttcccgcg

SEQ ID NO: 73 - DNA, 10 bp, Artificial Sequence
  E2F decoy core sequence
taaagggcgc

SEQ ID NO: 74 - DNA, 9 bp, Artificial Sequence
  E2F decoy core sequence
tttcccgcg

SEQ ID NO: 75 - DNA, 9 bp, Artificial Sequence
  E2F decoy core sequence
aaagggcgc

SEQ ID NO: 76 - DNA, 17 bp, Artificial Sequence
  E2F decoy molecule
ctagtttcccgcggatc

SEQ ID NO: 77 - DNA, 17 bp, Artificial Sequence
  E2F decoy molecule
gatcaaagggcgcctag

(19) United States
(12) Patent Application Publication
(10) Pub. No.: US 2005/0164240 A1
(43) Pub. Date: Jul. 28, 2005

(54) E2F oligonucleotide decoy molecules

(75) Inventors: Christi Parham, Menlo Park, CA (US); Leslie McEvoy, Mountain View, CA (US); Jie Zhang, Campbell, CA (US); Xiaoping Rao, Cupertino, CA (US)

Correspondence Address: HELLER EHRMAN LLP, 275 MIDDLEFIELD ROAD, MENLO PARK, CA, 94025-3506, US

(73) Assignee: Corgentech, Inc., South San Francisco, CA (US)

(21) Appl. No.: 10/960,165

(22) Filed: Oct. 6, 2004

Related U.S. Application Data
(60) Provisional application No. 60/509,303, filed on Oct. 6, 2003.

(51) Int. Cl. C12Q 1/68

(52) U.S. Cl. 435/6

(57) ABSTRACT

The invention concerns E2F oligonucleotide decoy molecules with improved properties. In particular, the invention concerns a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from THP-1 cells.

### CROSS-REFERENCE TO RELATED APPLICATIONS

This is a non-provisional application filed under 37 C.F.R. 1.53(b), claiming priority under U.S.C. § 119(e) to provisional Application Ser. No. 60/509,303 filed Oct. 6, 2003.

### BACKGROUND OF THE INVENTION

1. Field of the Invention

The present invention concerns E2F oligonucleotide decoy molecules with improved properties.

2. Description of the Related Art

The E2F family of transcription factors plays a pivotal role in the control of cell cycle progression, and regulates the expression of numerous genes, including genes involved in cell cycle regulation, including those encoding c-Myc, c-Myb, Cdc2, proliferating-cell nuclear antigen (PCNA), Cyclin A, dihydrofolate reductase, thymidine kinase, and DNA polymerase α.

E2F is now recognized as a family of six heterodimeric complexes encoded by distinct genes, divided into two distinct groups: E2F proteins (E2F-1-E2F-6) and DP proteins (DP-1 and DP-2). The E2F proteins themselves can be divided into two functional groups, those that induce S-phase progression when over-expressed in quiescent cells (E2Fs 1-3), and those that do not (E2Fs 4-5). E2F-6 is functionally different in that its over-expression has been described to suppress the transactivational effects of co-expression of E2F-1 and DP-1. In addition, it has been reported that E2F-6 expression delays the exit from S-phase rather than inducing S-phase. The proteins from the E2F and DP groups heterodimerize to give rise to E2F activity. All possible combinations of E2F-DP complexes exist in vivo. Individual E2F-DP complexes invoke different transcriptional responses depending on the identity of the E2F moiety and the proteins that are associated with the complex. In addition homodimers of E2F molecules have also been described. (See, e.g. Zheng et al., Genes & Devel 13:666-674 (1999).)

Depending on whether they are associated with the retinoblastoma (Rb) family of pocket proteins, E2F proteins can act either as repressors or as activators of transcription (Hiebert et al. Genes & Devel 6:177-185 (1992); Weintraub et al., Nature 358:259-261 (2002)).

E2F transcription factors are responsible for activating a dozen or more genes that must be turned on during vascular cell growth and multiplication. Their blockade prevents abnormal cell proliferation (e.g. neointimal hyperplasia) that eventually result in atherosclerotic lesions. As a result of their biological functions, E2F transcription factors have been implicated in neointimal hyperplasia, neoplasia glomerulonephritis, angiogenesis, and inflammation.

Various members of the E2F family have also been described to play a role in cancer, and identified as targets for anti-cancer agents. For an overview of E2F family members, regulation and pathway see, e.g. Harbour, J. W., and Dean, D. C., Genes Dev 14, 2393-2409 (2000); Mundle, S. D., and Saberwal, G., Faseb J 17, 569-574 (2003); and Trimarchi, J. M., and Lees, J. A. Nat Rev Mol Cell Biol 3, 11-20 (2002).

E2F binding sites have been identified in the promoter regions of many cellular genes, and reported, for example, in the following publications: Farnham et al., Biochim. Biophys. Acta 1155:125-131 (1993); Nevins, J. R., Science 258:424-429 (1992); Shan et al., Mol. Cell. Biol. 14:299-309 (1994); Thalmeier et al., Genes Dev. 3:517-536 (1989); Delton et al., EMBO J. 11:1797-1804 (1992); Yamaguchi et al., Jpn. J. Cancer Res. 83:609-617 (1992).

Oligonucleotide decoys targeting E2F transcription factors have been described in PCT Publication No. WO 95/11687, published May 4, 1995, the entire disclosure of which is hereby expressly incorporated by reference.

E2F oligonucleotide decoys are in clinical development as a means of altering the natural history of vein grafts, without the potential hazards of methods that require the introduction of oligonucleotides in vivo, and are expected to be of great clinical value in solving a vexing problem confronting all surgical bypass and repair of arteries in a variety of clinical circumstances. The U.S. Food and Drug Administration has granted Fast Track designation for an E2F decoy molecule (Corgentech, Inc., South San Francisco, Calif.), which is designed to prevent blocking and failing of vein grafts used in coronary artery and peripheral arterial bypass procedures.

Further representative references concerning E2F decoy therapy include: Morishita, R., G. H. Gibbons, M. Horiuchi, K. E. Ellison, M. Nakama, L. Zhang, Y. Kaneda, T. Ogihara, and V. J. Dzau. (1995). A gene therapy strategy using a transcription factor decoy of the E2F binding site inhibits smooth muscle proliferation in vivo. Proceedings of the National Academy of Sciences USA, 92, 5855-5859; Dzau, V. J., M. J. Mann, R. Morishita, and Y. Kaneda. (1996). Fusigenic viral liposome for gene therapy in cardiovascular diseases. Proceedings of the National Academy of Sciences USA, 93, 11421-11425; von der Leyen, H. E., M. J. Mann, and V. J. Dzau. (1996). Gene inhibition and gene augmentation for the treatment of vascular proliferative disorders. Semin Interv Cardiology, 1, 209-214; Kaneda, Y., R. Morishita, and V. J. Dzau. (1997). Prevention of restenosis by gene therapy. Annals of the NY Academy of Sciences, 811, 299-308, discussion 308-210; Mann, M. J., and V. J. Dzau. (1997). Genetic manipulation of vein grafts. Current Opinion in Cardiology, 12, 522-527; Mann, M. J., G. H. Gibbons, P. S. Tsao, H. E. von der Leyen, J. P. Cooke, R. Buitrago, R. Kernoff, and V. J. Dzau. (1997). Cell cycle inhibition preserves endothelial function in genetically engineered rabbit vein grafts. Journal of Clinical Investigation, 99, 1295-1301; Morishita, R., G. H. Gibbons, M. Horiuchi, M. Nakajima, K. E. Ellison, W. Lee, Y. Kaneda, T. Ogihara, and V. J. Dzau. (1997). Molecular Delivery System for Antisense Oligonucleotides: Enhanced Effectiveness of Antisense Oligonucleotides by HVJ-liposome Mediated Transfer. Journal of Cardiovascular Pharmacology, 2, 213-222; Braun-Dullaeus, R. C., M. J. Mann, and V. J. Dzau. (1998). Cell cycle progression: new therapeutic target for vascular proliferative disease. Circulation, 98, 82-89; Mann, M. J. (1998). E2F decoy oligonucleotide for genetic engineering of vascular bypass grafts. Antisense Nucleic Acid Drug Development, 8, 171-176; Morishita, R., G. H. Gibbons, M. Horiuchi, Y. Kaneda, T. Ogihara, and V. J. Dzau. (1998). Role of AP-1 complex in angiotensin II-mediated transforming growth factor-beta expression and growth of smooth muscle cells: using decoy approach against AP-1 binding site. Biochemistry and Biophysics Res Community, 243, 361-367; Poston, R. S., K. P. Tran, M. J. Mann, E. G. Hoyt, V. J. Dzau, and R. C. Robbins. (1998). Prevention of ischemically induced neointimal hyperplasia using ex-vivo antisense oligodeoxynucleotides. Journal of Heart and Lung Transplant, 17, 349-355; Tomita, N., M. Horiuchi, S. Tomita, G. H. Gibbons, J. Y. Kim, D. Baran, and V. J. Dzau. (1998). An oligonucleotide decoy for transcription factor E2F inhibits mesangial cell proliferation in vitro. American Journal of Physiology, 275, F278-284; Mann, M. J., G. H. Gibbons, H. Hutchinson, R. S. Poston, E. G. Hoyt, R. C. Robbins, and V. J. Dzau. (1999). Pressure-mediated oligonucleotide transfection of rat and human cardiovascular tissues. Proceedings of the National Academy of Sciences USA, 96, 6411-6416; Mann, M. J., A. D. Whittemore, M. C. Donaldson, M. Belkin, M. S. Conte, J. F. Polak, E. J. Orav, A. Ehsan, G. Dell'Acqua, and V. J. Dzau. (1999). Ex-vivo gene therapy of human vascular bypass grafts with E2F decoy: the PREVENT single-centre, randomised, controlled trial. Lancet, 354, 1493-1498; Poston, R. S., M. J. Mann, E. G. Hoyt, M. Ennen, V. J. Dzau, and R. C. Robbins. (1999). Antisense oligodeoxynucleotides prevent acute cardiac allograft rejection via a novel, nontoxic, highly efficient transfection method. Transplantation, 68, 825-832; Tomita, S., N. Tomita, T. Yamada, L. Zhang, Y. Kaneda, R. Morishita, T. Ogihara, V. J. Dzau, and M. Horiuchi. (1999). Transcription factor decoy to study the molecular mechanism of negative regulation of renin gene expression in the liver in vivo. Circulation Research, 84, 1059-1066; von der Leyen, H. E., R. Braun-Dullaeus, M. J. Mann, L. Zhang, J. Niebauer, and V. J. Dzau. (1999). A pressure-mediated nonviral method for efficient arterial gene and oligonucleotide transfer. Human Gene Therapy, 10, 2355-2364; Ehsan, A., and M. J. Mann. (2000). Antisense and gene therapy to prevent restenosis. Vascular Medicine, 5, 103-114; Mann, M. J. (2000). Gene therapy for vein grafts. Current Cardiology Reports, 2, 29-33; Mann, M. J. (2000). Gene therapy for peripheral arterial disease. Molecular Medicine Today, 6, 285-291; Mann, M. J., and V. J. Dzau. (2000). Therapeutic applications of transcription factor decoy oligonucleotides. Journal of Clinical Investigation, 106, 1071-1075; Tomita, N., R. Morishita, S. Tomita, G. H. Gibbons, L. Zhang, M. Horiuchi, Y. Kaneda, J. Kaneda, J. Higaki, T. Ogihara, and V. J. Dzau. (2000). Long-term stabilization of vein graft wall architecture and prolonged resistance to experimental atherosclerosis after E2F decoy oligonucleotide gene therapy. Journal of Thoracic Cardiovascular Surgery, 121,714-722. The complete disclosures of the cited references are hereby expressly incorporated by reference.

### SUMMARY OF THE INVENTION

The invention concerns a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to the targeted E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from THP-1 cells.

In another aspect, the invention concerns a method for modulating the transcription of a gene that is regulated by E2F, comprising introducing into the nucleus of a cell containing such gene a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from Lps-stimulated THP-1 cells, in an amount sufficient to competitively inhibit the binding of E2F to the gene, whereby the transcription of said gene is modulated.

In a further aspect, the invention concerns a method for the prevention or treatment in a mammalian host of a disease or condition associated with E2F-regulated gene transcription, comprising introducing into the cells of the mammal a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from Lps-stimulated THP-1 cells, in an amount sufficient to competitively inhibit the binding of E2F to the gene, whereby the transcription of the gene is modulated.

The disease or condition associated with E2F regulated gene transcription can, for example, be coronary heart disease, peripheral vascular disease, arteriovenous graft failure, neointimal hyperplasia, proliferative disease, restenosis or cancer.

### BRIEF DESCRIPTION OF THE DRAWINGS

FIG. 1 shows the sequences for the “reference decoy molecule” (SEQ ID NOS 1 and 2), “novel decoy molecule” (SEQ ID NOS: 3 and 4) and “scrambled decoy molecule” (SEQ ID NOS 5 and 6), where the core sequences are bolded and underlined.

FIG. 2 shows the results of a competitive binding assay performed with a representative decoy molecule of the present invention, in comparison with a reference decoy and a negative control.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The terms “oligonucleotide decoy,” “double-stranded oligonucleotide decoy,” “oligodeoxynucleotide decoy,” and “double-stranded oligodeoxynucleotide decoy” are used interchangeably, and refer to short, double-stranded nucleic acid molecules, which bind to and interfere with a biological function of a targeted transcription factor. Accordingly, the terms “E2F oligonucleotide decoy,” “double-stranded E2F oligonucleotide decoy,” “E2F oligodeoxynucleotide decoy,” and “double-stranded E2F oligodeoxynucleotide decoy” are used interchangeably, and refer to short, double-stranded nucleic acid molecules, which bind to and interfere with a biological function of an E2F transcription factor.

The term “E2F” is used herein in the broadest sense and includes all naturally occurring E2F molecules of any animal species, including E2F-1, E2F-2, E2F-3, E2F-4, E2F-5, and E2F-6.

The term “transcription factor binding sequence” is a short nucleotide sequence to which a transcription factor binds. The term specifically includes naturally occurring binding sequences typically found in the regulatory regions of genes the transcription of which is regulated by one or more transcription factors. The term further includes artificial (synthetic) sequences, which do not occur in nature but are capable of competitively inhibiting the binding of the transcription factor to a binding site in an endogenous gene.

The term “double-stranded” is used to refer to a nucleic acid molecule comprising two complementary nucleotide strands connected to each other solely by Watson-Crick base pairing. The term specifically includes molecules which, in addition to the double-stranded region formed by the two complementary strands, comprise single-stranded overhang(s).

As used herein, the phrase “modified nucleotide” refers to nucleotides or nucleotide triphosphates that differ in composition and/or structure from natural nucleotides and nucleotide triphosphates.

As used herein, the terms “five prime” or “5′” and “three-prime” or “3′” refer to a specific orientation as related to a nucleic acid. Nucleic acids have a distinct chemical orientation such that their two ends are distinguished as either five-prime (5′) or three-prime (3′). The 3′ end of a nucleic acid contains a free hydroxyl group attached to the 3′ carbon of the terminal pentose sugar. The 5′ end of a nucleic acid contains a free hydroxyl or phosphate group attached to the 5′ carbon of the terminal pentose sugar.

As used herein, the term “overhang” refers to a double-stranded nucleic acid molecule, which does not have blunt ends, such that the ends of the two strands are not coextensive, and such that the 5′ end of one strand extends beyond the 3′ end of the opposing complementary strand. It is possible for a linear nucleic acid molecule to have zero, one, or two, 5′ overhangs.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured, for instance, by cell viability assays, FACS analysis or DNA electrophoresis.

The terms “cancer” and “cancerous” refer to or describe the physiological condition in mammals that is typically characterized by unregulated cell growth. Examples of cancer include, without limitation, carcinoma, lymphoma, leukemia, blastoma, and sarcoma. Specific examples of such cancers include squamous cell carcinoma, small-cell lung cancer, non-small cell lung cancer, breast cancer, pancreatic cancer, glioblastoma multiforme, cervical cancer, stomach cancer, bladder cancer, hepatoma, colon carcinoma, and head and neck cancer. In a preferred embodiment, the cancer includes breast cancer, ovarian cancer, prostate cancer, and lung cancer.

The term “mammal” as used herein refers to any animal classified as a mammal, including humans, higher primates, cows, horses, dogs and cats. In a preferred embodiment of the invention, the mammal is a human.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

B. Detailed Description

Design of E2F Decoys with Improved Properties

It is well known that many transcription factors can bend the DNA upon binding to their recognition site and that nonlinear DNA structures facilitate and even determine proximal and distal DNA-protein contacts involved in transcription (Perez-Martin et al., Microbiol Rev 58:268-290 (1994); and van der Vliet and Verrijzer, Bioassays 15:25-32 (1993)). More recently, the E2F recognition site has been found to contain an intrinsic DNA bend (Cress and Nevins, Mol. Cel.. Biol. 16:2119-2127 (1996)). The binding of free E2F to this recognition site results in a DNA bend similar in magnitude to the intrinsic bend but in the opposite orientation. It is also known that the structure of the E2F-1 promoter affects the transcriptional activity of the promoter. Five base-pair substitutions in and around the E2F site change the DNA helix structure, E2F binding and influence transcriptional activity. The natural bend in the E2F binding sites together with the fact that E2F binding to the site has a dramatic effect on this structure seems to suggest a role for DNA structure in E2F binding and E2F-dependent transcriptional control. Binding of transcription factors to their binding sites is sensitive to the structure and shape of the DNA. The level of specificity of interaction is enhanced by flexibility and/or distortion in the DNA. For further details see, also Philos Trans R Soc Lond B Biol Sci 351:501-9 (1996) and Rhodes et al., Indian J. Biochem Biophys 33:83-7 (1996).

The present invention, in part, is based on the finding that by changing the shape and/or structure of an E2F decoy molecule, one can greatly improve its binding affinity to the target E2F transcription factor, which, in turn results in more effective inhibition of the biological function of the target E2F transcription factor. The invention is further based on identification of a minimum sequence needed for E2F binding.

As part of the present invention, the shape/structure of the E2F decoy molecule has been changed by changing the sequences flaking the core binding sequence, which resulted in an order of a magnitude improvement in E2F binding affinity. The increased binding affinity makes the E2F decoy a much more potent inhibitor of E2F biological function. The shape and structure of the DNA are influenced by the base pair sequence, length of the DNA, backbone and nature of the nucleotide (i.e. native DNA vs. modified sugars or bases). Thus, the shape and/or structure of the molecule can also be changed by other approaches, such as, for example, by changing the total length, the length of the fully complementary, double-stranded region within the molecule, by alterations within the core and flanking sequences, by changing the backbone structure and by base modifications. E2F decoy molecules having increased binding affinity and/or improved in vivo stability can be designed and made by any of such approaches or by any combinations thereof.

In particular, by changing the core sequence (including its length, sequence, base modifications and backbone structure) it is possible to change the binding affinity, the stability and the specificity of the E2F decoy molecule. Changes in the flanking sequence have a genuine impact on and can significantly increase the in vivo stability of the molecule, and may affect binding affinity and/or specificity

Thus, in its broadest aspect, the invention concerns E2F decoy double-stranded oligodeoxynucleotide (dsODN) molecules, that have a flexible structure capable of changing shape and/or structure, e.g. bending, and have increased binding affinity to the target E2F transcription factor or factors. Thus, the E2F decoy molecules of the present invention can have increased binding affinity to one or more of E2F- 1, E2F-2, E2F-3, E2F-4, E2F-5, and E2F-6.

In a more specific aspect, the present invention concerns E2F decoy double-stranded oligodeoxynucleotide (dsODN) molecules with improved properties. In particular, the invention concerns novel E2F decoy dsODN molecules, which have high binding affinity for an E2F transcription factor (including its heterodimer (E2F/DP) and homodimer (E2F/E2F) forms) and/or exhibit improved stability in vivo.

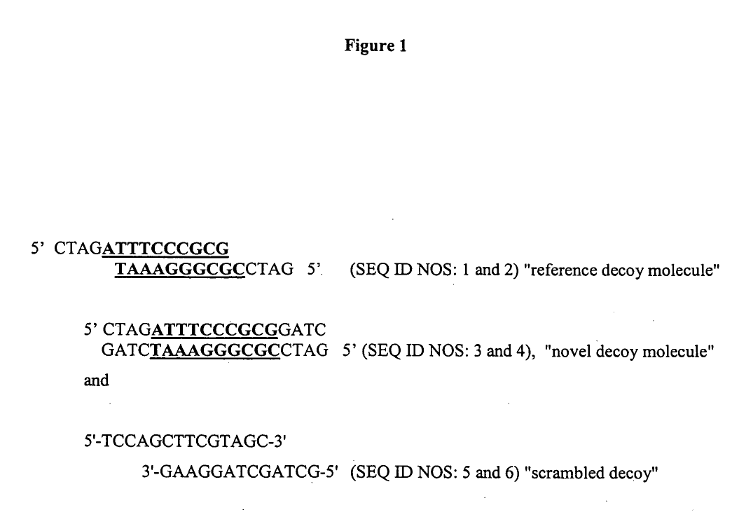

In one embodiment, the E2F decoy dsODN molecule comprises a core sequence that is capable of specific binding to an E2F transcription factor, flanked by 5′ and 3′ sequences, wherein (i) the core sequence consists of about 5 to 12, preferably about 6 to 10 base pairs; (ii) the molecule comprises an about 12 to 28, preferably about 14 to 24 base-pair long double-stranded region composed of two fully complementary strands; and (iii) the E2F decoy dsODN binds to the target E2F transcription factor with a binding affinity that is at least about 5-fold, or at least about 7-fold, or at least about 10-fold, or at least about 15-fold of the binding affinity of the reference decoy molecule of FIG. 1 (SEQ ID NOs: 1 and 2), as determined by a competitive gel mobility shift assay performed on nuclear extract from vascular smooth muscle cells (VSMCs), following the protocol described in Example 1. Preferably, the melting temperature (Tm) of the improved E2F decoy dsODN molecule is also significantly higher than the Tm of the reference decoy molecule of FIG. 1 (SEQ ID NOs: 1 and 2) (42.3° C.).

The length of the fully-complementary double-stranded portion of the E2F decoy molecule herein is believed to be important for enhanced binding affinity and stability. In order to achieve these improved properties, this region should contain at least about 12 base pairs, and typically its length is between about 12 and about 28 base pairs. The “fully complementary” region consists of two nucleotide strands where each nucleotide in the first strand undergoes Watson-Crick base pairing with each nucleotide in the second strand.

The core sequence typically should comprise at least 6 base pairs, and usually at least about 8 base pairs for satisfactory binding to the target E2F transcription factor. Generally, the core sequence consists of about 5 to 12, more typically about 6 to 10 base pairs. The core sequence may be or may contain sequences from the E2F binding sequences in the promoter region of a gene, the transcription of which is up- or down-regulated by an E2F transcription factor. Alternatively, the core sequence may be a synthetic sequence that does not occur in nature as an E2F binding sequence, such as a consensus sequence that is designed based on the nucleotide at each site which occurs most frequently in the E2F binding sequences of various genes, or binding sequences for various E2F transcription factors.

The flanking sequences are typically about 5 to 50 bases long, and can be, but need not be, fully complementary. Thus, the flanking region(s) may comprise single stranded overhangs at either end. It is believed that binding affinity and stability are affected more by the length and sequence of the truly double-stranded region, composed of two fully complementary strands within the oligonucleotide decoy molecules of the present invention than by the length of the flanking region(s) per se.

The nucleotide sequences present in the decoy molecules of the present invention may comprise modified or unusual nucleotides, and may have alternative backbone chemistries. Synthetic nucleotides may be modified in a variety of ways, see, e.g. Bielinska et al. Science 25);997 (1990). Thus, oxygens may be substituted with nitrogen, sulfur or carbon; phosphorys substituted with carbon; deoxyribose substituted with other sugars, or individual bases substituted with an unnatural base. In each case, any change will be evaluated as to the effect of the modification on the binding ability and affinity of the oligonucleotide decoy to the E2F trascription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g. Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β, D-mannosylqueosine, 5-methoxycarbonylmethyl-2-thiouridine, 5-metoxycarbonalmethyluridine, 5-methoxyuridine, 2-methylthio-N6-isopentenyladenosine, N-((9-beta-D-ribofuransyl-2-methylthiopurine-6-yl)carbamoyl)threonine, N-((9-beta-D-ribofuranosylpurine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuransylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl.

The E2F decoy molecules of the present invention can be synthesized by standard phosphodiester or phosphoramidate chemistry, using commercially available automatic synthesizers.

In a particular embodiment, the E2F decoy molecules of the present invention include a core sequence comprising a strand selected from the group consisting of

| | |
|---|---|
| TTTSGCGS | (SEQ ID NO: 7) |
| TTTGGCGC | (SEQ ID NO: 8) |
| TTTCGCGC | (SEQ ID NO: 9) |
| TTTCCCGC | (SEQ ID NO: 10) |
| TTTGCCGC | (SEQ ID NO: 11) |
| CTTCCCGC | (SEQ ID NO: 12) |
| GTTCCCGC | (SEQ ID NO: 13) |
| CTTCGCGC | (SEQ ID NO: 14) |
| TTAGCGCC | (SEQ ID NO: 15) |
| TGAGCGCC | (SEQ ID NO: 16) |
| GTAGCGCC | (SEQ ID NO: 17) |
| GGAGCGCC | (SEQ ID NO: 18) |
| CTAGCGCC | (SEQ ID NO: 19) |
| CGAGCGCC | (SEQ ID NO: 20) |
| GTTCGCGC | (SEQ ID NO: 21) |
| TTTGCGCC | (SEQ ID NO: 22) |
| TGTGCGCC | (SEQ ID NO: 23) |
| GTTGCGCC | (SEQ ID NO: 24) |
| GGTGCGCC | (SEQ ID NO: 25) |
| CTTGCGCC | (SEQ ID NO: 26) |
| CGTGCGCC | (SEQ ID NO: 27) |
| TTTCCGG | (SEQ ID NO: 28) |
| TTTCGCGG | (SEQ ID NO: 29) |
| GTTGGCGC | (SEQ ID NO: 30) |
| CTTGGCGC | (SEQ ID NO: 31) |
| CTTGCCGC | (SEQ ID NO: 32) |
| GTTGCCGC | (SEQ ID NO: 33) |
| TTTGGCGG | (SEQ ID NO: 34) |
| TTACCGCC | (SEQ ID NO: 35) |
| TGACCGCC | (SEQ ID NO: 36) |
| GTACCGCC | (SEQ ID NO: 37) |
| GGACCGCC | (SEQ ID NO: 38) |
| CTACCGCC | (SEQ ID NO: 39) |
| CGACCGCC | (SEQ ID NO: 40) |
| TTTCCGCC | (SEQ ID NO: 41) |
| TGTCCGCC | (SEQ ID NO: 42) |
| GTTCCGCC | (SEQ ID NO: 43) |
| GGTCCGCC | (SEQ ID NO: 44) |
| CTTCCGCC | (SEQ ID NO: 45) |
| CGTCCGCC | (SEQ ID NO: 46) |
| CTTCCCGG | (SEQ ID NO: 47) |
| TTTGCCGG | (SEQ ID NO: 48) |
| GTTCCCGG | (SEQ ID NO: 49) |
| CTTCGCGG | (SEQ ID NO: 50) |
| TTTGCGCG | (SEQ ID NO: 51) |
| TTAGCGCG | (SEQ ID NO: 52) |
| TGTGCGCG | (SEQ ID NO: 53) |
| TGAGCGCG | (SEQ ID NO: 54) |
| GTTGCGCG | (SEQ ID NO: 55) |
| GTAGCGCG | (SEQ ID NO: 56) |
| GGTGCGCG | (SEQ ID NO: 57) |
| TTTSGCGCGMNR | (SEQ ID NO: 58) |
| CTTGGCGG | (SEQ ID NO: 59) |
| GTTGGCGG | (SEQ ID NO: 60) |
| GTTCGCGG | (SEQ ID NO: 61) |
| TTTCCCGG | (SEQ ID NO: 62) |
| CTTGCCGG | (SEQ ID NO: 63) |
| GTTGCCGG | (SEQ ID NO: 64) |
| TGTCGCGC | (SEQ ID NO: 65) |
| CTTCCCGG | (SEQ ID NO: 66) |
| CGTCGCGC | (SEQ ID NO: 67) |
| GGTCGCGC | (SEQ ID NO: 68) |
| TTTCGGGC | (SEQ ID NO: 69) |
| TGTGGCGC | (SEQ ID NO: 70) |
| TGTCGCGG | (SEQ ID NO: 71) |

and its complement where S
is a G or a C:

Based in this information and other knowledge about the structure of intrinsic E2F binding sites, one skilled in the art can further optimize the structure of the E2F decoy molecules herein, for example, by known techniques of molecular modeling, co-crystallization with the E2F-DP complex, and other means known in the art. The actual sequence of the flaking regions near the core sequence is more critical than the sequence of more distant regions. Thus, the identity of the nucleotides at positions adjacent to or within a few nucleotides from the core sequence needs to be more carefully controlled than the identity of the nucleotides at positions farther away from the core sequence. In a particular embodiment, the flanking sequences are those shown in FIG. 1, SEQ ID NOS: 3 and 4, which can be coupled with any of the core sequences listed above.

As discussed earlier, E2F and DP proteins form heterodimers to give rise to E2F functional activity. In addition, homodimers of certain E2F proteins have also been described. Individual E2F-DP or E2F-E2F species invoke different transcriptional responses depending on the identity of the E2F moiety and the proteins that are associated with the complex. If desired, the E2F dsODN molecules can designed to exhibit preferential binding to one or more E2F transcription factors, which, in turn, is expected to result in different in vivo biological activities. Thus, E2F decoy molecules useful in cancer therapy can be designed by this approach.

The binding affinity of a candidate decoy molecule can be determined by standard methods, for example, by a gel shift mobility assay. The gel shift, or electrophoretic mobility shift (EMSA), assay provides a rapid and sensitive method for detecting the binding of transcription factors, or other DNA-binding proteins, to DNA. The assay is based on the observation that complexes of protein and DNA migrate through a non-denaturing polyacrylamide gel more slowly than free double-stranded oligonucleotides. The gel shift assay is performed by incubating a purified protein, or a complex mixture of proteins (such as nuclear extracts), with a ³²p end-labeled DNA fragment containing the transcription factor-binding site. The reaction products are then analyzed on a nondenaturing polyacrylamide gel. The specificity of the transcription factor for the binding site is established by competition experiments using excess amounts of oligonucleotides either containing a binding site for the protein of interest or a scrambled DNA sequence. The identity of proteins contained within a complex is established by using an antibody which recognizes the protein and then looking for either reduced mobility of the DNA-protein-antibody complex or disruption of the binding of this complex to the radiolabeled oligonucleotide probe.

Prime Therapeutic Targets

The E2F decoy molecules of the present invention are expected to find clinical use in the prevention and treatment of coronary heart disease, the single leading killer of American men and women, that caused over 450,000 deaths in the United States in 1998, according to the American Heart Association.

In addition, E2F decoys find utility in the treatment of peripheral vascular disease, which is characterized by atherosclerotic narrowing of peripheral arteries and, as a result, adversely affects blood circulation. In early clinical stages, the disease manifests itself in leg pain, but if left untreated, it can develop into gangrene, necessitating amputation of the limb, and substantial and irreversible morbidity and mortality.

A further clinical target for E2F decoys is neointimal hyperplasia, the pathological process that underlies graft atherosclerosis, stenosis, and the majority of vascular graft occlusion. Neointimal hyperplasia is commonly seen after various forms of vascular injury, and is a major component of the vein graft's response to harvest and surgical implantation into high-pressure arterial circulation.

In addition, an important role for E2F in the development of cancer has been suggested. As discussed earlier, E2F is responsible for inducing expression of a group of genes required for cell growth and cell division. When the cell receives growth inhibitory signals, E2F is inactivated by the tumor suppressor retinoblastoma gene, Rb. As a result, the growth control genes regulated by E2F remain inactive and the cell is held in a quiescent state. It has been proposed that in tumor cells which carry mutated copies of Rb, E2F is no longer controlled by Rb. As a result, E2F activates the genes directing cell division and so leaves the cell in a permanently proliferative state. For further details, including alternative mechanisms, see, e.g. Johnson and Schneider-Broussard, Front Biosci 3:d447-8 (1998). It has been reported that small peptides which inhibit E2F activity when introduced into tumor cell lines cause apoptosis (Bandara et al., Nature Biotechnology 15:896-901 (1997). Regardless of the underlying mechanism, the E2F decoy molecules of the present invention hold promise in the treatment of various types of cancer including breast cancer.

Administration of the E2F Decoys

A preferred mode of delivering the E2F decoys of the present invention is pressure-mediated transfection, as described, for example, in U.S. Pat. Nos. 5,922,687 and 6,395,550, the entire disclosures of which are hereby expressly incorporated by reference. In brief, the E2F decoy molecules are delivered to cells in a tissue by placing the decoy nucleic acid in an extracellular environment of the cells, and establishing an incubation pressure around the cells and the extracellular environment. The establishment of the incubation pressure facilitates the uptake of the nucleic acid by the cells, and enhances localization to the cell nuclei.

More specifically, a sealed enclosure containing the tissue and the extracellular environment is defined, and the incubation pressure is established within the sealed enclosure. In a preferred embodiment, the boundary of the enclosure is defined substantially by an enclosing means, so that target tissue (tissue comprising the target cell) is subjected to isotropic pressure, and does not distend or experience trauma. In another embodiment, part of the enclosure boundary is defined by a tissue. A protective means such as an inelastic sheath is then placed around the tissue to prevent distension and trauma in the tissue. While the incubation pressure depends on the application, incubation pressures about 300 mmHg-1500 mmHg above atmospheric pressure, or at least about 100 mmHg above atmospheric pressure are generally suitable for many applications.

The incubation period necessary for achieving maximal transfection efficiency depends on parameters such as the incubation pressure and the target tissue type. For some tissue, such as human vein tissue, an incubation period on the order of minutes (>1 minute) at low pressure (about 0.5 atm) is sufficient for achieving a transfection efficiency of 80-90%. For other tissue, such as rat aorta tissue, an incubation period on the order of hours (>1 hour) at high pressure (about 2 atm) is necessary for achieving a transfection efficiency of 80-90%.

Suitable mammalian target tissue for this type of delivery includes blood vessel tissue (in particular veins used as grafts in arteries), heart, bone marrow, and connective tissue, liver, genital-urinary system, bones, muscles, gastrointestinal organs, and endocrine and exocrine organs. A method of the present invention can be applied to parts of an organ, to a whole organ (e.g. heart), or to a whole organism. In one embodiment a nucleic acid solution can be perfused into a target region (e.g. a kidney) of a patient, and the patient is subject to pressure in a pressurization chamber. Specific applications include the treatment of allografts (grafts derived from a different subject than the transplant patient) and syngrafts (grafts derived from the transplant patient).

For other applications, the E2F decoys of the present invention can be administered by other conventional techniques. For example, retrovial transfection, transfection in the form of liposomes are among the known methods suitable for transfection. For details see also Dzau et al., Trends in Biotech 11:205-210 (1993); or Morishita et al., Proc. Natl. Acad. Sci. USA 90:8474-8478 (1993). Where administered in liposomes, the decoy concentration in the lumen will generally be in the range of about 0.1 μM to about 50 μM per decoy, more usually about 1 μM to about 10 μM, most usually about 3 μM.

For other delivery techniques, the most suitable concentration can be determined empirically. The determination of the appropriate concentrations and doses is well within the competence of one skilled in the art. Optimal treatment parameters will vary depending on the indication, decoy, clinical status of the patient, etc., and can be determined empirically based on the instructions provided herein and general knowledge in the art.

The decoys may be administered as compositions comprising individual decoys or mixtures of decoys. Usually, a mixture contains up to 6, more usually up to 4, more usually up to 2 decoy molecules.

Further details of the invention will be apparent from the following non-limiting Examples.

### EXAMPLE 1

Competitive Binding Study

1. Synthesis of Oligonucleotide Decoys

The double-stranded oligonucleotide decoy molecules shown in FIG. 1 have been synthesized using an automated DNA synthesizer (Model 380B; Applied Biosystems, Inc., Foster City, Calif.). The decoys were purified by column chromatography, lyophilized, and dissolved in culture medium. Concentrations of each decoy were determined spectrophotometrically.

The double-stranded oligonucleotide molecule represented by SEQ ID NOS: 1 and 2 (the “reference decoy molecule”) is a known decoy, currently in clinical development. The double-stranded oligonucleotide decoy represented by SEQ ID NOS: 3 and 4 (the “novel decoy molecule”) is a variant with significantly improved properties, while the “scrambled decoy” represented by SEQ ID NOS: 5 and 6 is used as a negative control.

The Tm of the novel decoy molecule is 55° C., significantly higher than the 42.3° C. Tm of the reference molecule. As a result, the novel decoy molecule is expected to be far more stable in vivo that the reference decoy.

2. Competitive Gel Mobility-Shift Assay

The difference in the ability of the novel decoy molecule to compete with the reference decoy and the negative control (scrambled decoy) to compete for E2F binding to a labeled probe containing an E2F consensus binding site was tested in vitro in LPS-stimulated THP-1 cells essentially following the protocol described in Morishita et al., Proc. Natl. Acad. Sci. USA 92:5855-5859 (1995). Nuclear extract was prepared from vascular smooth muscle cells (VSMCs) as described in Horiuchi et al., J. Biol. Chem. 266:16247-16254 (1991). A gel shift mobility assay was performed as follows:

A double-stranded oligonucleotide containing the E2F binding site (5′ CTAGATTTCCCGCGGATC 3′) (SEQ ID NO: 3) was end-labeled with γ³²P-ATP using T4 Polynucleotide Kinase (Promega). Five μg of a nuclear extract prepared from LPS stimulated THP-1 cells was incubated with 50 fmol of radiolabeled probe in the presence or absence of competing novel decoy molecule, the reference decoy or the negative control (scrambled decoy). The incubations were carried out at room temperature for 30 minutes in a 20 μl reaction volume composed of 10 mM Tris PH7.4, 40 mM KCL, 1 mM DTT, 0.1 mM EDTA, 8% Glycerol, 0.05% NP-40 and 0.5 μg Poly-dIdC. The reactions were loaded onto a 6% polyacrylamide gel, subjected to electrophoresis and dried. The dried gels were imaged and quantitated using a Typhoon 8600 PhosphorImager (Amersham) and ImageQuant software. The identity of the E2F proteins contained in complexes bound to the radiolabeled oligonucleotide probe were identified by pre-incubating the reactions for 5 minutes with individual antibodies specific for each member of the E2F family prior to the addition of the radiolabeled probe. Antibodies against E2F1(sc-193x, sc-251x), E2F2 (sc-633x), E2F3 (sc-878x, sc-879x), E2F4 (sc-866x), E2F5 (sc-999x), p107(sc-318x) and cyclinA (sc-239x) were purchased from Santa Cruz Biotechnology.

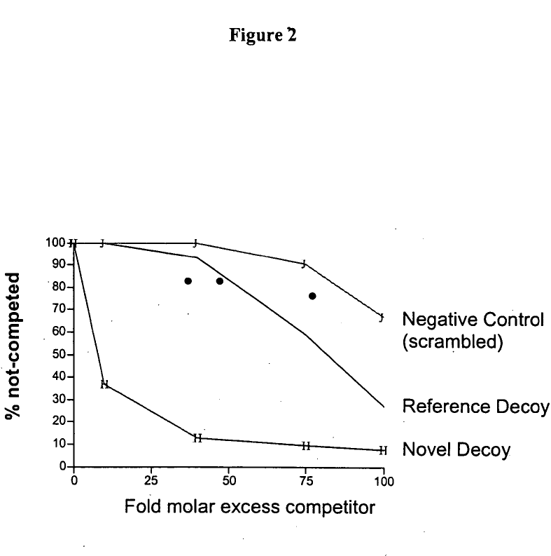

As shown in FIG. 2, the novel decoy molecule was able to compete with binding of a labeled probe with E2F in the smooth muscle extracts by greater than 60% at 10-fold molar excess (compared to 7% blockade by the reference decoy), and by 90% at 40-fold molar excess (compared to only 40% by the reference decoy). Thus, the novel decoy molecule is approximately a magnitude better competitor than the reference decoy molecule of the prior art.

### EXAMPLE 2

Determination of Minimal Sequence for E2F Binding

The structural requirements of the core binding site for E2F were examined by generating mutations of the core sequence of the E2F decoy molecules herein, and testing their ability to block binding to the consensus E2F binding sequence. Binding was assessed using the TransFactor™ method plate assays (BD Clontech, Palo Also, Calif.). Briefly, double-stranded oligonucleotides containing the E2F consensus binding sequence were immobilized on 96-well plates. E2F family members were cloned and expressed in E. coli, and crude bacterial lysates were incubated in the wells of the plate at 30° C. for 4.5 hours, to allow the transcription factors to bind to the oligonucleotide on the plate. The amount of E2F present was quantitated using an antibody specific for the particular E2F family member being assayed. Detection was performed using a secondary antibody conjugated to horseradish peroxidase and spectroscopic quantification. The E2F decoy or decoys with mutations in the core binding site were added (in increasing molar amounts over the bound oligonucleotide) as a competitor for binding of the E2F family members away from the bound oligonucleotide. The greater the reduction in bound transcription factor, the more competitive is the decoy molecule assayed. A scrambled oligonucleotide molecule (SEQ ID NOS: 5 and 6) was added in the same molar amounts as the E2F decoy tested, to assess specificity of the binding.

It was determined that on the upper strand in the core sequence the nucleotides TTTCCCGCG (SEQ ID NO: 74) were required for E2F binding. The first nucleotide “A” in SEQ ID NO: 72, was not required for E2F binding. Mutation of the complementary “T” on the 3′ end of the bottom strand core sequence (SEQ ID NO: 73) gave the same result. Based on these results, the following core sequence is sufficient for E2F binding:

| | |
|---|---|
| 5′-TTTCCCGCG | (SEQ ID NO: 74) |
| AAAGGGCGC-5′ | (SEQ ID NO: 75) |

All references cited throughout the specification are hereby expressly incorporated by reference.

Although the invention is illustrated by reference to certain embodiments, it is not so limited. Further variations in the design, sequence, making and use of the E2F decoy molecules herein are possible and are within the skill of an ordinary artisan. All such modifications are intended to be specifically within the scope of the invention.

1. A double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from Lps-stimulated THP-1 cells.

2. The E2F decoy molecule of claim 1 wherein the core sequence consists of about 6 to 10 base pairs.

3. The E2F decoy molecule of claim 2 wherein the core sequence is 10 base pairs long.

4. The E2F decoy molecule of claim 1 wherein the core sequence consists of about 6 to 9 base pairs.

5. The E2F decoy molecule of claim 1 wherein the core sequence is 9 base pairs long.

6. The E2F decoy molecule of claim 1 wherein the core sequence comprises a strand having a sequence selected from the group consisting of

| | |
|---|---|
| TTTSGCGS | (SEQ ID NO: 7) |
| TTTGGCGC | (SEQ ID NO: 8) |
| TTTCGCGC | (SEQ ID NO: 9) |
| TTTCCCGC | (SEQ ID NO: 10) |
| TTTGCCGC | (SEQ ID NO: 11) |
| CTTCCCGC | (SEQ ID NO: 12) |
| GTTCCCGC | (SEQ ID NO: 13) |
| CTTCGCGC | (SEQ ID NO: 14) |
| TTAGCGCC | (SEQ ID NO: 15) |
| TGAGCGCC | (SEQ ID NO: 16) |
| GTAGCGCC | (SEQ ID NO: 17) |
| GGAGCGCC | (SEQ ID NO: 18) |
| CTAGCGCC | (SEQ ID NO: 19) |
| CGAGCGCC | (SEQ ID NO: 20) |
| GTTCGCGC | (SEQ ID NO: 21) |
| TTTGCGCC | (SEQ ID NO: 22) |
| TGTGCGCC | (SEQ ID NO: 23) |
| GTTGCGCC | (SEQ ID NO: 24) |
| GGTGCGCC | (SEQ ID NO: 25) |
| CTTGCGCC | (SEQ ID NO: 26) |
| CGTGCGCC | (SEQ ID NO: 27) |
| TTTCCGG | (SEQ ID NO: 28) |
| TTTCGCGG | (SEQ ID NO: 29) |
| GTTGGCGC | (SEQ ID NO: 30) |
| CTTGGCGC | (SEQ ID NO: 31) |
| CTTGCCGC | (SEQ ID NO: 32) |
| GTTGCCGC | (SEQ ID NO: 33) |
| TTTGGCGG | (SEQ ID NO: 34) |
| TTACCGCC | (SEQ ID NO: 35) |
| TGACCGCC | (SEQ ID NO: 36) |
| GTACCGCC | (SEQ ID NO: 37) |
| GGACCGCC | (SEQ ID NO: 38) |
| CTACCGCC | (SEQ ID NO: 39) |
| CGACCGCC | (SEQ ID NO: 40) |
| TTTCCGCC | (SEQ ID NO: 41) |
| TGTCCGCC | (SEQ ID NO: 42) |
| GTTCCGCC | (SEQ ID NO: 43) |
| GGTCCGCC | (SEQ ID NO: 44) |
| CTTCCGCC | (SEQ ID NO: 45) |
| CGTCCGCC | (SEQ ID NO: 46) |
| CTTCCCGG | (SEQ ID NO: 47) |
| TTTGCCGG | (SEQ ID NO: 48) |
| GTTCCCGG | (SEQ ID NO: 49) |
| CTTCGCGG | (SEQ ID NO: 50) |
| TTTGCGCG | (SEQ ID NO: 51) |
| TTAGCGCG | (SEQ ID NO: 52) |
| TGTGCGCG | (SEQ ID NO: 53) |
| TGAGCGCG | (SEQ ID NO: 54) |
| GTTGCGCG | (SEQ ID NO: 55) |
| GTAGCGCG | (SEQ ID NO: 56) |
| GGTGCGCG | (SEQ ID NO: 57) |
| TTTSGCGCGMNR, | (SEQ ID NO: 58) |
| CTTGGCGG | (SEQ ID NO: 59) |
| GTTGGCGG | (SEQ ID NO: 60) |
| GTTCGCGG | (SEQ ID NO: 61) |
| TTTCCCGG | (SEQ ID NO: 62) |
| CTTGCCGG | (SEQ ID NO: 63) |
| GTTGCCGG | (SEQ ID NO: 64) |
| TGTCGCGC | (SEQ ID NO: 65) |
| CTTCCCGG | (SEQ ID NO: 66) |
| CGTCGCGC | (SEQ ID NO: 67) |
| GGTCGCGC | (SEQ ID NO: 68) |
| TTTCGGGC | (SEQ ID NO: 69) |
| TGTGGCGC | (SEQ ID NO: 70) |
| TGTCGCGG | (SEQ ID NO: 71) |

and its complement.

7. The E2F decoy molecule of claim 1 wherein the core sequence is

| | |
|---|---|
| ATTTCCCGCG | (SEQ ID NOS: 72 and 73) |
| TAAAGGGCGC. | |

8. The E2F decoy molecule of claim 1 wherein the core sequence is

| | |
|---|---|
| TTTCCCGCG | (SEQ ID NOS: 74 and 75) |
| AAAGGGCGC. | |

9. The E2F decoy molecule of claim 1 comprising an about 14 to 24 base-pair long double-stranded region composed of two fully complementary strands.

10. The E2F decoy molecule of claim 1 binding to said E2F with a binding affinity that is at least about 7-fold of the binding affinity of said reference decoy molecule.

11. The E2F decoy molecule of claim 1 binding to said E2F with a binding affinity that is at least about 10-fold of the binding affinity of said reference decoy molecule.

12. The E2F decoy molecule of claim 1 binding to said E2F with a binding affinity that is at least about 15-fold of the binding affinity of said reference decoy molecule.

13. The E2F decoy molecule of claim 1 comprising single-stranded overhangs.

14. The E2F decoy molecule of claim 1 wherein the two strands are associated with each other by Watson-Crick base pairing, in the absence of a covalent bond.

15. The E2F decoy molecule of claim 1 which has a melting temperature (Tm) higher than the melting temperature of said reference decoy molecule.

16. The E2F decoy molecule of claim 15 which has a Tm of at least about 50 ° C.

17. The E2F decoy molecule of claim 15 which has a Tm of at least about 55 ° C.

18. The E2F decoy molecule of claim 1 which is

| | |
|---|---|
| 5′ CTAGATTTCCCGCGGATC | (SEQ ID NOS: 3 and 4) |
| GATCTAAAGGGCGCCTAG 5′. | |

19. The E2F decoy molecule of claim 1 which is

| | |
|---|---|
| 5′ CTAGTTTCCCGCGGATC | (SEQ ID NOS: 76 and 77) |
| GATCAAAGGGCGCCTAG 5′. | |

20. A method for modulating the transcription of a gene that is regulated by E2F, comprising introducing into the nucleus of a cell containing said gene a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from Lps-stimulated THP-1 cells, in an amount sufficient to competitively inhibit the binding of E2F to said gene, whereby the transcription of said gene is modulated.

21. The method of claim 20 which is performed in vivo.

22. The method of claim 20 which is performed ex vivo.

23. The method of claim 20 wherein said E2F decoy molecule is capable of episomal replication in said cell.

24. The method of claim 20 wherein said E2F decoy is delivered as a composition.

25. The method of claim 24 wherein said composition comprises liposomes.

26. The method of claim 25 wherein said liposomes comprise lipid and a viral coat protein.

27. The method of claim 20 wherein said E2F decoy is introduced into the nucleus of said cell by pressure-mediated transfection.

28. The method of claim 20 wherein said cells are vascular smooth muscle cells, tumor cells or endothelial cells.

29. A method for the prevention or treatment in a mammalian host of a disease or conditions associated with E2F-regulated gene transcription, comprising introducing into the cells of said mammal a double-stranded E2F decoy oligodeoxynucleotide (dsODN) molecule comprising a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a reference decoy molecule shown in FIG. 1 (SEQ ID NOS: 1 and 2), as determined by a competitive gel mobility shift binding assay performed on nuclear extract from Lps-stimulated THP-1 cells, in an amount sufficient to competitively inhibit the binding of E2F to said gene, whereby the transcription of said gene is modulated.

30. The method of claim 29 wherein said disease or condition is selected from the group consisting of coronary heart disease, peripheral vascular disease, arteriovenous graft failure, neointimal hyperplasia, proliferative disease, restenosis and cancer.

31. The method of claim 30 wherein said disease or condition is a cancer.
